(19) United States
(12) Patent Application Publication
(10) Pub. No.: US 2025/0235394 A1
(43) Pub. Date: Jul. 24, 2025

(54) BIOFILM-TARGETING NANOPARTICLES TO INCREASE THE ANTICARIES EFFECT OF FLUORIDE

(71) Applicant: The Regents of The University of Michigan, Ann Arbor, MI (US)

(72) Inventors: Livia M.A. TENUTA, Saline, MI (US); Joerg LAHANN, Ann Arbor, MI (US); Albert CHANG, Ann Arbor, MI (US); Nahal HABIBI, Ann Arbor, MI (US)

(73) Assignee: The Regents of The University of Michigan, Ann Arbor, MI (US)

(21) Appl. No.: 18/711,268

(22) PCT Filed: Nov. 18, 2022
(86) PCT No.: PCT/US2022/050426

§ 371 Date: May 17, 2024

Related U.S. Application Data
(60) Provisional application No. 63/281,202, filed on Nov. 19, 2021.

(51) Int. Cl. A61K 8/66; A61K 8/19; A61K 8/21; A61K 8/24; A61Q 11/00

(52) U.S. Cl. CPC A61K 8/66; A61K 8/19; A61K 8/21; A61K 8/24; A61Q 11/00

(57) ABSTRACT

Nanoparticles for treating a tooth in an oral cavity of a subject are provided. The nanoparticle comprises a biocompatible and biodegradable hydrophilic polymer, a matrix-degrading enzyme, and an anticaries active ingredient present in the nanoparticle at greater than or equal to about 20% by weight. The nanoparticle has a zeta potential between about −10 mV to about +10 mV at a pH of 7. The nanoparticle is capable of selectively accumulating within a biofilm matrix associated with a surface of the tooth in the oral cavity of the subject. Oral care composition and methods of treating a tooth in an oral cavity of a subject with such nanoparticles are also provided.

### CROSS-REFERENCE TO RELATED APPLICATIONS

This application claims the benefit of U.S. Provisional Application No. 63/281,202, filed on Nov. 19, 2021. The entire disclosure of the above application is incorporated herein by reference.

### FIELD

The present disclosure relates to biofilm-targeting nanoparticles having anticaries agents that can be delivered to surfaces within an oral cavity of a subject to provide long-term therapeutic treatment of caries.

### BACKGROUND

This section provides background information related to the present disclosure which is not necessarily prior art.

Oral biofilms are formed on various surfaces in the mouth and include dental biofilms where microorganisms are bound to surfaces of teeth. Dental caries (i.e., dental decay) is a leading health concern worldwide, which develop when dental biofilms are formed in the presence of fermentable sugars. The biofilm initially forms when a pellicle of glycoproteins is formed over the tooth enamel or dentin. This is followed by attachment and embedding of microorganisms therein. Various microorganisms, including many species of bacteria, are held together by a matrix of extracellular polymers, including polysaccharides, lipids, proteins, and nucleic acids. Thus, the extracellular matrix is comprised of polysaccharides produced from some type of sugars, extracellular DNA from dead bacteria, and other macromolecules from bacteria or saliva origin that gives structure to the biofilm. Caries or dental cavities form when bacteria in the biofilm matrix ferment sugars and produce acids, which demineralize enamel and/or dentin. Early decalcification is indicated by white spot lesions or microcavities forming on the enamel surface. Microcavities or carious lesions may be reversed by remineralization early in the process. However, should decalcification continue, irreversible cavitation in the tooth occurs with the caries, requiring a dental procedure to stop further decalcification.

The caries process can be prevented or arrested by the use of certain preventive methods. The most effective conventional methods are based on fluoride use (fluoridated water, fluoride in toothpaste and mouth rinses, fluoride in clinical or professionally available products, like gels or varnishes), which are considered to be responsible for visible decline in caries levels. Fluoride as an effective anticaries agent can reduce the loss of tooth minerals under a cariogenic attack, increase remineralization of early caries lesions, and reduce bacterial metabolism (reducing acid production) at high concentrations. All of the above anticaries effects require the ion to be free (ionic, F⁻) in the oral fluids (saliva, dental biofilm fluid) and are local (topical) in the oral cavity.

When fluoride toothpastes and rinses are used, there is not a prolonged retention of the fluoride in the mouth, especially in the dental biofilm (under which caries lesions are formed). The biofilm on teeth is negatively charged so that negatively charged fluoride is prevented from being retained in the negatively charged biofilm. Thus, fluoride clearance occurs because there are no binding mechanisms for fluoride to be retained; it is an anion and repelled by a predominance of negative charges in dental biofilm. Therefore, most of the fluoride that is used for brushing/rinsing is lost. Therefore, fluoride used in high concentrations in oral care compositions achieves only a modest clinical effectiveness considering the very high fluoride concentration. Further, current fluoride-containing toothpastes and rinses do not provide a high enough fluoride concentration to form calcium fluoride deposits (as a longer-term fluoride reservoir for a continuous release of fluoride ions over longer periods of time).

Dental caries in certain patients can pose particular challenges. Due to its aggressive nature, it can be problematic when caries affects so-called high caries-risk groups, such as older adults, by way of non-limiting example. Older adults who take medications that cause dry mouth suffer from aggressive caries progression, especially in the roots of teeth. Hyposalivation escalates the rate of caries progression, especially for root surfaces. Root caries is expected to increase in aging populations worldwide. Unfortunately, there is no specific treatment developed to control root caries in such challenging situations.

Thus, while there are a variety of methods for preventing and treating caries, these generally involve administering high concentrations of fluoride that are rapidly cleared from the mouth. It would be desirable to have a method of preventing or minimizing dental caries which is easily administered and assessed, has substantivity on the target dental surfaces, is biocompatible and has low toxicity, and/or can dissolve or disintegrate in vivo at predetermined time intervals to provide targeted treatment.

### SUMMARY

This section provides a general summary of the disclosure, and is not a comprehensive disclosure of its full scope or all of its features.

In certain aspects, the present disclosure contemplates a composition for oral administration. The composition may comprise a nanoparticle. The nanoparticle comprises an active ingredient (e.g., a therapeutic agent). In certain variations, a biofilm-targeting nanoparticle for treating a tooth in an oral cavity of a subject is provided. The biofilm-targeting nanoparticle is capable of selectively accumulating within a biofilm matrix associated with a surface of the tooth in the oral cavity of the subject. The nanoparticle comprises a biocompatible and biodegradable hydrophilic polymer, a matrix-degrading enzyme, and an anticaries active ingredient. The anticaries active ingredient is present in the nanoparticle at greater than or equal to about 20% by weight. Further, the nanoparticle has a zeta potential between about −10 mV to about +10 mV at a pH of 7.

In certain aspects, the anticaries active ingredient is selected from the group consisting of: a fluoride-containing compound, a calcium-containing compound, a phosphate-containing compound, and/or a calcium and phosphate-containing compound.

In certain aspects, the anticaries active ingredient is present at greater than or equal to about 25% to less than or equal to about 70% by weight of the nanoparticle.

In certain aspects, the anticaries active ingredient comprises a fluoride salt.

In certain aspects, the anticaries active ingredient is selected from the group consisting of: calcium fluoride, sodium fluoride, magnesium fluoride, strontium fluoride, stannous fluoride, sodium monofluorophosphate, amine fluoride, calcium-phosphate compounds, and combinations thereof.

In certain aspects, the anticaries active ingredient comprises calcium fluoride and is present at greater than or equal to about 45% by weight after incorporation into the nanoparticle.

In certain aspects, the anticaries active ingredient comprises stannous fluoride and is present at greater than or equal to about 45% by weight after incorporation into the nanoparticle.

In certain aspects, the biocompatible and biodegradable hydrophilic polymer is selected from the group consisting of: chitosan, gelatin A, cationic dextran, pectin, dextrin, poly(amidoamine), poly(2-N,N-dimethylaminoethylmethacrylate), and combinations thereof.

In certain aspects, the nanoparticle is configured to selectively accumulate within the biofilm matrix associated with the surface of the tooth for greater than or equal to about 1 day.

In certain aspects, the nanoparticle has an average diameter of greater than or equal to about 10 nm to less than or equal to about 500 nm.

In certain aspects, the matrix-degrading enzyme is selected from the group consisting of: dextranase, mutanase, nucB, and combinations thereof.

In certain aspects, the biocompatible and biodegradable hydrophilic polymer is selected from the group consisting of: chitosan, gelatin A, cationic dextran, poly(amidoamine), poly(2-N,N-dimethylaminoethylmethacrylate), and combinations thereof. Further, the anticaries active ingredient is selected from the group consisting of: calcium fluoride, sodium fluoride, magnesium fluoride, strontium fluoride, stannous fluoride, sodium monofluorophosphate, amine fluoride, calcium-phosphate compounds, and combinations thereof. The matrix-degrading enzyme is selected from the group consisting of: dextranase, mutanase, nucB, and combinations thereof.

In certain aspects, the biocompatible and biodegradable hydrophilic polymer comprises chitosan having a molecular weight (Mₙ) of greater than or equal to about 30 kDa to less than or equal to about 375 kDa. The anticaries active ingredient comprises calcium fluoride and is present at greater than or equal to about 45% by weight after incorporation into the nanoparticle to less than or equal to about 60% by weight. The matrix-degrading enzyme comprises dextranase.

In other aspects, the present disclosure provides an oral care composition for treating a tooth in an oral cavity of a subject. The oral care composition comprises a plurality of biofilm-targeting nanoparticles. The plurality of biofilm-targeting nanoparticles is capable of selectively accumulating within a biofilm matrix associated with a surface of the tooth in the oral cavity of the subject. Each nanoparticle comprises a biocompatible and biodegradable hydrophilic polymer, a matrix-degrading enzyme, and an anticaries active ingredient. The anticaries active ingredient is present in the nanoparticle at greater than or equal to about 20% by weight. Each nanoparticle of the plurality of biofilm-targeting nanoparticles has a zeta potential between about −10 mV to about +10 mV at a pH of 7. Further, the oral care composition has an orally acceptable carrier comprising water. The plurality of nanoparticles is distributed in the orally acceptable carrier.

In certain aspects, the oral care composition is selected from the group consisting of: a mouth rinse, a dentifrice, a gel, a varnish, a paint, a lozenge, a troch, chewing gum, a chewing tablet, intraoral film, adhesive strip, a pellet or bead for adhering to teeth, a sponge or pellet, chewing gum, and a chewing tablet.

In certain aspects, the biocompatible and biodegradable hydrophilic polymer is selected from the group consisting of: chitosan, gelatin A, pectin, dextrin, cationic dextran, poly(amidoamine), poly(2-N,N-dimethylaminoethylmethacrylate), and combinations thereof. Further, the anticaries active ingredient is selected from the group consisting of: calcium fluoride, sodium fluoride, magnesium fluoride, strontium fluoride, stannous fluoride, sodium monofluorophosphate, amine fluoride, calcium-phosphate compounds, and combinations thereof. The matrix-degrading enzyme is selected from the group consisting of: dextranase, mutanase, and combinations thereof.

In certain aspects, the biocompatible and biodegradable hydrophilic polymer comprises chitosan having a molecular weight (number weight) of greater than or equal to about 30 kDa to less than or equal to about 375 kDa, the anticaries active ingredient comprises calcium fluoride and is present at greater than or equal to about 45% by weight after incorporation into the nanoparticle to less than or equal to about 60% by weight, and the matrix-degrading enzyme comprises dextranase.

In certain aspects, the oral care composition is a mouth rinse and the anticaries active ingredient is calcium fluoride or stannous fluoride.

In yet other aspects, the present disclosure provides a method of preventing or treating caries in a tooth in an oral cavity of a subject. The method may comprise introducing a plurality of biofilm-targeting nanoparticles into the oral cavity of the subject to associate with the tooth. Each nanoparticle of the plurality of biofilm-targeting nanoparticles comprises a biocompatible and biodegradable hydrophilic polymer, a matrix-degrading enzyme, and an anticaries active ingredient present in the nanoparticle at greater than or equal to about 20% by weight. Each nanoparticle has a zeta potential between about −10 mV to about +10 mV at a pH of 7.

In certain aspects, the anticaries active ingredient comprises a fluoride salt that is configured to deliver greater than or equal to about 0.1 ppm to less than or equal to about 10 ppm of fluoride ions for greater than or equal to about 1 day to the biofilm matrix associated with the surface of the tooth.

In certain aspects, the anticaries active ingredient has substantivity on the biofilm matrix associated with the surface of the tooth for greater than or equal to about 1 day to less than or equal to about 1 week.

In certain aspects, the biocompatible and biodegradable hydrophilic polymer is selected from the group consisting of: chitosan, gelatin A, cationic dextran, poly(amidoamine), poly(2-N,N-dimethylaminoethylmethacrylate), and combinations thereof. Further, the anticaries active ingredient is selected from the group consisting of: calcium fluoride, sodium fluoride, magnesium fluoride, strontium fluoride, stannous fluoride, sodium monofluorophosphate, amine fluoride, calcium-phosphate compounds, and combinations thereof. The matrix-degrading enzyme is selected from the group consisting of: dextranase, mutanase, and combinations thereof.

In certain aspects, the biocompatible and biodegradable hydrophilic polymer comprises chitosan having a molecular weight (number weight) of greater than or equal to about 30 kDa to less than or equal to about 375 kDa. The anticaries active ingredient comprises calcium fluoride. The anticaries active ingredient is present at greater than or equal to about 45% by weight to less than or equal to about 60% by weight after incorporation into the nanoparticle. Further, the matrix-degrading enzyme comprises dextranase.

In certain aspects, the biofilm-targeting nanoparticles are capable of selectively accumulating within a biofilm matrix associated with a surface of the tooth in the oral cavity of the subject.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 shows an effect of the nanoparticles prepared in accordance with certain variations of the present disclosure (ChitCaF₂NP with dextranase (Dex)). On a side designated A of FIG. 1 (left side), fluoride from currently available treatments is shown to have limited capacity to bind to dental biofilm. On a side designated B of FIG. 1 (right side), enhanced availability of fluoride in dental biofilm is shown for certain variations of the current technology. A dextranase component (not shown) facilitates penetration of chitosan-CaF₂ nanoparticles (Chit-CaF₂np) prepared in accordance with certain aspects of the present disclosure into the biofilm, where the nanoparticles are retained due to their mildly neutral net charge for continuous fluoride release. Fluoride reduces bacterial acid production and potentiates mineral gain by the tooth (reducing demineralization and enhancing remineralization).

FIGS. 2A-2C. Characterization and fluoride release of nanoparticles prepared in accordance with certain variations of the present disclosure (ChitCaF₂NP). FIG. 2A shows SEM image of particles, where zeta potential is 3.81 mV (DLS analysis). Particle composition: High molecular weight chitosan (310-375 kDa) (1% w/v). FIG. 2B shows size analysis (insert: log scale). Size: 68.9+57.5 nm; PDI: 0.176. FIG. 2C shows cumulative fluoride released from the Chit-CaF₂NP. Methods: Fluoride release of Chit-CaF₂NP suspension (1.5×10¹¹ particles/mL) or CaF₂ nanoparticles (Nanoshel LLC, DE) to DPBS through a dialysis membrane at 37° C.

FIG. 3. Shows inhibition of acidogenicity by nanoparticles prepared in accordance with certain variations of the present disclosure (ChitCaF₂NP). Methods: S. mutans were treated with solutions with increasing fluoride concentrations (NaF) or with a suspension of the ChitCaF₂NP. After treatment removal and washing, cells were resuspended in TSB medium containing 1% glucose, and pH was assessed over time. * ChitCaF₂NP differ significantly from the 22.6 ppm F group at 60 and 90 min (p<0.05). Average±SD, n=4.

FIG. 4 shows binding of nanoparticles prepared in accordance with certain variations of the present disclosure (ChitCaF₂NP) to S. mutans. S. mutans cells were treated with NaF solutions (0, 22.6, or 226 ppm F) or with a ChitCaF₂NP suspension prepared in accordance with certain aspects of the present disclosure (22.6 ppm F). Unbound particles separated from pellets by centrifugation at 500×g; control tubes containing only ChitCaF₂NP were used to discount fluoride coming from unbound precipitated particles (35.7±1.8%). Total fluoride in the bacterial pellets was extracted using 0.5 M HCl. Different letters show differences between treatments at p<0.0001. Average±SD, n=4.

FIG. 5 shows increased fluoride retention in dental biofilm by the chitosan-calcium fluoride nanoparticles (“ChitCaF₂np”) and dextranase (Dex) therapy. Methods: 2-day-old S. mutans biofilms were treated with different levels of dextranase (Dex, 0, 1 or 20 U/mL in sodium acetate buffer, pH 5.5) for 15 min at 37° C., and then with increasing fluoride concentrations (NaF) for comparison or the ChitCaF₂nps for 30 minutes. Biofilms were washed with PBS and collected for determination of fluoride concentration. Effect of dextranase is significant only for the ChitCaF₂np (p<0.01). Mean±SD, n=2.

FIG. 6 shows a proposed binding mechanism for nanoparticles prepared in accordance with certain variations of the present disclosure (ChitCaF₂NP). The figure shows protonation of negatively charged groups and release of the Chit-CaF₂np during a pH drop.

FIGS. 7A-7B show reduced fluoride bioavailability and enhanced safety of nanoparticles prepared in accordance with certain variations of the present disclosure (ChitCaF₂NP). In FIG. 7A, fluoride in the femur of rats treated with 226 ppm F (NaF or ChitCaF₂NP) or deionized water is shown. FIG. 7B shows histopathological analysis of the lateral border of the tongue in a ChitCaF₂NP-treated animal (H&E, 10×).

FIGS. 8A-8D show an effect of an enzyme for degrading the EPS matrix (like dextranase (Dex) on nanospheres penetrating into biofilms. Biofilms treated with placebo (FIGS. 8A, 8C) or dextranase (FIGS. 8B, 8D) followed by the addition of nanospheres. Red=S. mutans 3209/pVMCherry; Blue=EPS; Green=fluorescent nanospheres. In FIG. 8B, large accumulations of nanospheres are clearly visible penetrating into biofilm pores. FIGS. 8C and 8D are each from a confocal stack where each image is located deep on the biofilm (approximately 2.5 μm above the bottom). In FIG. 8D, many spheres are in intimate contact with and within the biofilms (the green channel shows 14.8 times more pixels in D than in C (Image J)). Scale bars=20 μm.

FIG. 9 shows a percentage of fluoride released from fast-release nanoparticles comprising stannous fluoride prepared in accordance with certain variations of the present disclosure (ChitSnF₂NP) versus time (hours) when in an aqueous solution.

FIGS. 10A-10B. FIG. 10A shows a graph of a dose-response of an experimental tooth model to fluoride at increasing concentrations (22.6, 226 and 2,260 ppm F) on the % of surface hardness loss (% SHL) in the enamel slabs. FIG. 10B shows a comparison of anticaries effect by treatment with 226 ppm F where 100% is NaF compared to a composition having 226 ppm F with 10% of fluoride in the form of nanoparticles prepared in accordance with certain variations of the present disclosure (ChitCaF₂NP) and 90% of fluoride as NaF.

FIG. 11 shows fluoride concentration (micromoles F/g biofilm wet weight) in an S. mutans biofilm 16 hours after the last treatment with solutions containing 22.6, 226 and 2,260 ppm F or 226 ppm F with 10% of fluoride in the form of nanoparticles prepared in accordance with certain variations of the present disclosure (ChitCaF₂NP) and 90% of fluoride as NaF.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters.

In addition, disclosure of ranges includes disclosure of all values and further divided ranges within the entire range, including endpoints and sub-ranges given for the ranges.

The disclosure of all patents, patent applications, articles, and other publications referenced or cited in this disclosure are hereby incorporated by reference herein.

Example embodiments will now be described more fully with reference to the accompanying drawings.

Caries includes various stages of bacterial decay of a tooth and enamel demineralization, including initial microscopic increases in pore size in the tooth enamel (e.g., microcavity development) to extensive decay and cavities leading to severe loss of tooth structure and eventually loss of the tooth. The characteristic feature of active carious lesions in enamel with decalcification, when dry, is a white and rough surface. This indicates an increase in microscopic pore size of the enamel. An active lesion is one that is progressing toward cavitation (demineralizing). Subsurface demineralization may eventually cause collapse of the overlying tooth surface, creating cavitation.

Prevention and treatment of dental caries often involves use of fluoride as an anti-caries agent. However, after use of fluoride products in the mouth, fluoride—a negatively charged ion—is not retained for long periods of time because the oral environment is predominantly negatively charged. For example, fluoride and the biofilm formed on surfaces in the oral cavity, like teeth, are both negatively charged. Thus, negatively charged fluoride is prevented from penetrating and being retained on teeth by the negatively charged biofilm. The main mechanism of action of fluoride to control caries is by being available in the oral fluids. However, fluoride is quickly washed away from the mouth due to the action of saliva and further being repelled by negative surfaces present in the oral cavity. As such, in conventional oral care products high fluoride concentrations are delivered, which can risk overexposure to fluoride that can be toxic. Moreover, although fluoride is still the cornerstone of caries prevention, few innovations to improve its anticaries efficacy have been made.

In various aspects, the present disclosure provides nanoparticles that can bind to a dental biofilm and/or other negatively charged intraoral surface, for an increased retention for a given concentration of fluoride. The present disclosure thus contemplates an innovative treatment to boost the effect of fluoride by delivering neutrally charged nanoparticles capable of releasing fluoride that are engineered to penetrate and bind to a biofilm (e.g., an exopolysaccharide (EPS)-rich dental biofilm) on the surface of a tooth in an oral cavity, such as cariogenic dental plaque. The penetration into the cariogenic biofilm mass is enhanced by the present of an enzymatic component (e.g., dextranase) that breaks extracellular polymers, facilitating the inward diffusion of the nanoparticles. The nanoparticles enable localized delivery of high fluoride levels in dental plaque or the biofilm matrix, without increasing the level of exposure to the fluoride ion (and hence potential toxicity) and can reduce caries, including root caries progression in individuals with dry mouth. It was believed that neutral nanoparticles were generally considered incapable of penetrating through a biofilm, for example, one published study showed that neutral nanoparticles become stationary at the biofilm matrix and cannot penetrate it. However, the nanoparticles prepared in accordance with certain aspects of the present disclosure do at least partially penetrate the biofilm matrix and serve to cloak the negative charge of the fluoride ions, so that fluoride is delivered locally to the tooth surface. This improved anticaries approach is thus able to reduce the cariogenicity of dental biofilm and control root caries. For example, this can help control rampant caries progression in high caries-risk groups, such as older adults suffering from hyposalivation. In certain aspects, the nanoparticles may further be capable of interacting with negatively charged surfaces in the oral cavity, including enamel, dentin, roots, emerging carious lesions and cavities at the tooth surface or within the tooth.

In certain aspects, the present disclosure provides an oral care composition for minimizing, preventing, or treating carious lesions in teeth in the oral cavity of a subject. The oral care composition may be administered to the subject, which may be a human, companion animal, such as a cat, dog, or horse, and the like. The oral care composition may comprise a plurality of nanoparticles. Each nanoparticle may include a biocompatible and biodegradable hydrophilic polymer.

By “biocompatible,” it is meant that a material or combination of materials can be in contact with cells, tissue in vitro or in vivo, or used with a subject (such as mammals or other organisms) and has acceptable toxicological properties for contact and/or beneficial use with such cells, tissue, and/or animals. For instance, a biocompatible material may be one that is suitable for administration in a subject without adverse consequences, for example, without substantial toxicity or acute or chronic inflammatory response and/or acute rejection of the material by the immune system, for instance, via a T-cell response. It will be recognized, of course, that “biocompatibility” is a relative term, and some degree of inflammatory and/or immune response is to be expected even for materials that are highly compatible with living tissue. However, non-biocompatible materials are typically those materials that are highly toxic, inflammatory and/or are acutely rejected by the immune system, e.g., a non-biocompatible material implanted into a subject may provoke an immune response in the subject that is severe enough such that the rejection of the material by the immune system cannot be adequately controlled, in some cases even with the use of immunosuppressant drugs, and often can be of a degree such that the material must be removed from the subject. In certain aspects, biocompatible materials are those that are approved for use in humans by an appropriate regulatory agency, such as the Federal Drug Administration (FDA) in the United States; the European Commission (EC)/European Medicines Agency (EMEA) in Europe; or Health Products and Food Branch (HPFB) in Canada.

By “biodegradable,” in certain aspects, the material dissolves or disintegrates at different rates ex vivo or in vivo. Dissolving refers to physical disintegration, erosion, disruption and/or dissolution of a material and may include the resorption of a material by a living organism. The polymeric material or other component forming the nanoparticle may dissolve or disintegrate at different rates or have different solubility (e.g., aqueous solubility) that impacts the rate of dissolution. The materials can dissolve or erode upon exposure to a solvent comprising a high concentration of water, such as saliva, serum, growth or culture media, blood, or bodily fluids. Disintegration may also include the material breaking into small pieces, which may collectively form a colloid or gel. In certain variations, the nanoparticle (and its polymer) degrades in a time period of greater than or equal to about 1 hour, optionally greater than or equal to about 4 hours, optionally greater than or equal to about 8 hours, optionally greater than or equal to about 12 hours, optionally greater than or equal to about 18 hours, optionally greater than or equal to about 24 hours (1 day), optionally greater than or equal to about 36 hours, and in certain variations, optionally greater than or equal to about 48 hours (2 days). In other variations, the nanoparticle may degrade in less than or equal to about 30 days after introduction into the oral cavity and exposure to saliva, optionally in less than or equal to about 14 days, optionally in less than or equal to about 7 days, optionally in less than or equal to about 5 days, optionally in less than or equal to about 3 days, optionally in less than or equal to about 2 days, and in certain variations, optionally in less than or equal to about 1 day. An oral composition may have a degradation time of greater than or equal to about 30 minutes to less than or equal to about 30 days after introduction into the oral cavity, optionally greater than or equal to about 60 minutes to less than or equal to about 14 days, optionally greater than or equal to about 4 hours to less than or equal to about 7 days, and in certain variations, optionally greater than or equal to about 2 days to less than or equal to about 7 days.

In certain aspects, the material may be “bio-based,” meaning that at least a substantial portion, for example 50% or more, of a material is made from one or more substances derived from living or once-living organisms. A bio-based material may comprise biopolymers, which are polymers produced by living organisms or derived from polymers produced by living organisms. Nanoparticles include particles optionally made up either partially or entirely of organic materials.

The nanoparticle thus comprises a polymer, such as a biocompatible, biodegradable, and/or bio-based polymer that is hydrophilic. In certain aspects, nanoparticles may partially or entirely comprise cross-linked polymers, which might, in some cases, be a single molecule. A “hydrophilic” polymer is one that is soluble in water or other aqueous (e.g., polar) solutions. Hydrophilic polymers may have one or more polar or functional groups that facilitate solubility in water or aqueous solutions. The nanoparticles are thus water-soluble or dispersible. As noted above, the nanoparticle itself is desirably at a substantially neutral charge. This may result in a cumulative or net charge of all the respective components in the nanoparticle being neutral. For example, a nanoparticle may comprise one or more of a polymer, an active ingredient (e.g., a therapeutic anti-caries agent), and a matrix-degrading enzyme, where one or more of these constituents has a negative charge and/or negative charge (such as negatively charged fluoride balancing any positive charges on a cationic hydrophilic polymer), but the nanoparticle still exhibits a neutral charge capable of associating with a cariogenic biofilm on a tooth.

In certain aspects, the neutrally charged nanoparticle has a net charge or zeta potential value at the pH of saliva (a pH of about 7) that is greater than or equal to about −10 mV to less than or equal to about +10 mV, optionally greater than or equal to about −8 mV to less than or equal to about +8 mV, optionally greater than or equal to about −7 mV to less than or equal to about +7 mV, optionally greater than or equal to about −6 mV to less than or equal to about +6 mV, optionally greater than or equal to about −5 mV to less than or equal to about +5 mV, optionally greater than or equal to about −4 mV to less than or equal to about +4 mV, optionally greater than or equal to about −3 mV to less than or equal to about +3 mV, and in other variations greater than or equal to about +2 mV to less than or equal to about +2 mV. In certain variations, the nanoparticle having such ranges of zeta potential is thus capable of associating with the negatively charged biofilm on the surface of a tooth. In one variation, the nanoparticle or component may have a net neutral charge and/or a zeta potential between about −10 mV to about +10 mV at a pH of 7.

Without intending to be limited by any particular theory, it is believed that the net neutral charge of the nanoparticle in combination with the biofilm matrix-degrading enzyme helps to penetrate and form openings in the biofilm matrix on the tooth surface to permit the nanoparticle to be associated with and/or embedded in the biofilm matrix that has a negative charge on the tooth surface. In this manner, the nanoparticle advantageously has substantivity on the tooth surface at the biofilm matrix level, where it can provide concentrated and sustained release of anticaries or other active ingredients. In accordance with various aspects of the present disclosure, the particles attach, adhere, and/or adsorb to oral components to increase the substantivity of fluoride in the mouth. In certain aspects, the nanoparticle is delivered to the mouth and binds to one or more of the following: oral surfaces (hard and soft tissue), teeth, biofilms (e.g., dental plaque), and anionic surfaces (e.g., enamel, dentin, carious lesions, and cavities).

“Substantivity” is generally defined as the prolonged association between a material and a substrate in the oral cavity (e.g., oral mucosa, oral proteins, dental plaque, dental surface) where an association is greater and more extended than would be expected from a simple deposition mechanism. In certain aspects, the nanoparticles of the present disclosure may have substantivity with various oral surfaces in the oral cavity. For example, the nanoparticles of the present disclosure may have substantivity with various negatively-charged oral surfaces in the oral cavity. This substantivity and retention of the nanoparticles on the oral surface (e.g., tooth surface) is especially advantageous where the anticaries agent comprises a fluoride-releasing compound, because fluoride ions are negatively charged and retention or substantivity on the oral surface (e.g., tooth surface) is challenging due to both the tooth surface/biofilm matrix and fluoride both sharing negative charges that repel one another.

Thus, nanoparticles provided by various aspects of the present disclosure provide fluoride therapy that increases the substantivity of fluoride, prolonging its effect in the mouth under the use of similar total fluoride concentrations. By increasing the substantivity of fluoride, when nanoparticles are introduced into the oral cavity and contacted with surfaces of teeth, a substantially increased fluoride effect helps to diminish dental caries. Advantageously, because fluoride is retained in the mouth and slowly released, the fluoride therapy provided in accordance with certain aspects of the present disclosure is also expected to be safer than other fluoride treatments; by increasing substantivity, reduced amounts of fluoride are necessary, therefore reducing the risk of chronic fluoride toxicity (dental fluorosis).

In certain aspects, the nanoparticles provided in accordance with the present disclosure are capable of preventing, minimizing, or treating one or more carious lesions when the nanoparticles are associated with a surface of the subject's tooth, for example, by at least partially penetrating or embedding in the biofilm matrix. In this manner, the nanoparticles provide localized and sustained release of the active agent, such as fluoride ions, at the surface of the teeth where the cariogenic bacteria are present and active. In certain variations, the nanoparticle may be designed to provide different release profiles, such as fast release of fluoride ions, slow release of fluoride ions, continuous release from the nanoparticle (for example, an initially high release rate followed by a continuously slow release over time afterwards).

When the nanoparticles provided by various aspects of the present disclosure are associated with the surface in the oral cavity, such as the tooth surface, a local concentration of fluoride ions at the tooth surface may be greater than or equal to about 0.1 part per million (ppm) to less than or equal to about 10 ppm in certain variations, optionally greater than or equal to about 1 part per million (ppm) to less than or equal to about 10 ppm.

Notably, the duration of retention on the tooth surface may correspond to the degradation rate of the nanoparticle, which in turn relates to the degradation rate of the hydrophilic polymer in the nanoparticle. In certain aspects, the nanoparticle may have substantivity or be retained locally in the biofilm matrix at the tooth surface for greater than or equal to about 1 hour, optionally greater than or equal to about 4 hours, optionally greater than or equal to about 8 hours, optionally greater than or equal to about 12 hours, optionally greater than or equal to about 18 hours, optionally greater than or equal to about 24 hours (1 day), optionally greater than or equal to about 36 hours, and in certain variations, optionally greater than or equal to about 48 hours (2 days). In other variations, the nanoparticle may be retained on the tooth surface in the biofilm matrix for less than or equal to about 30 days after introduction into the oral cavity and exposure to saliva, optionally for less than or equal to about 14 days (2 weeks), optionally for less than or equal to about 7 days (1 week), optionally for less than or equal to about 5 days, optionally for less than or equal to about 3 days, optionally for less than or equal to about 2 days, and in certain variations, optionally for less than or equal to about 1 day.

In yet other variations, the nanoparticle may be retained on the tooth surface in the biofilm matrix for less than or equal to about 30 days after introduction into the oral cavity and exposure to saliva for greater than or equal to about 30 minutes to less than or equal to about 30 days after introduction into the oral cavity, optionally greater than or equal to about 60 minutes to less than or equal to about 14 days (2 weeks), optionally greater than or equal to about 4 hours to less than or equal to about 7 days (1 week), optionally greater than or equal to about 1 day to less than or equal to about 7 days, and in certain variations, optionally greater than or equal to about 2 days to less than or equal to about 7 days.

In certain variations, the biocompatible and biodegradable hydrophilic polymer may be a polymer selected from the group consisting of: a mono-, oligo-, or polysaccharide, such as chitosan, gelatin (e.g., gelatin A), dextran, pectin, dextrin, poly(amidoamine) (PAA), poly(2-N,N-dimethylaminoethylmethacrylate) PDMAEMA, combinations and equivalents thereof. In certain particular variations, the biocompatible and biodegradable hydrophilic polymer comprises chitosan having a molecular weight (number average molecular weight (Mₙ)) of greater than or equal to about 30 kDa to less than or equal to about 375 kDa. In certain variations, the chitosan may have a molecular weight of greater than or equal to about 30 kDa to less than or equal to about 120 kDa, optionally greater than or equal to about 40 kDa to less than or equal to about 110 kDa, and in certain variations, optionally greater than or equal to about 50 kDa to less than or equal to about 100 kDa. In other variations, the chitosan may have a higher molecular weight, for example, from greater than or equal to about 310 kDa to less than or equal to about 375 kDa.

The term “nano-sized” or “nanometer-sized” as used herein is generally understood to be less than or equal to about 1 micrometer (i.e., 1,000 nanometers). Thus, the nanoparticle has at least one spatial dimension that is less than about 1 μm, optionally less than or equal to about 750 nm, optionally less than about 500 nm, and in certain aspects, less than about 200 nm. In certain aspects, all spatial dimensions of the nanoparticle component are less than or equal to about 1 μm (1,000 nm).

In certain aspects, the nanoparticles of the present disclosure have an average particle size or diameter of less than or equal to about 1 micrometer (1,000 nanometers—nm). In certain aspects, the average diameter of the nanoparticle may be greater than or equal to about 1 nm to less than or equal to about 1,000 nm, optionally greater than or equal to about 10 nm to less than or equal to about 1,000 nm, optionally greater than or equal to about 20 nm to less than or equal to about 1,000 nm, optionally greater than or equal to about 30 nm to less than or equal to about 1,000 nm, optionally greater than or equal to about 50 nm to less than or equal to about 1,000 nm, optionally greater than or equal to about 100 nm to less than or equal to about 1,000 nm, optionally greater than or equal to about 10 nm to less than or equal to about 900 nm, optionally greater than or equal to about 100 nm to less than or equal to about 900 nm, optionally greater than or equal to about 10 nm to less than or equal to about 800 nm, optionally greater than or equal to about 100 nm to less than or equal to about 800 nm, optionally greater than or equal to about 10 nm to less than or equal to about 500 nm, optionally greater than or equal to about 100 nm to less than or equal to about 500 nm, optionally greater than or equal to about 10 nm to less than or equal to about 300 nm, optionally greater than or equal to about 50 nm to less than or equal to about 300 nm, optionally greater than or equal to about 100 nm to less than or equal to about 300 nm, and in certain variations, optionally greater than or equal to about 200 nm to less than or equal to about 300 nm.

In some aspects, the nanoparticles may have an average size that is less than the size of the pores formed by the biofilm matrix-degrading enzyme on the biofilm matrix on the surface of the tooth. The size of such pores may vary, but are generally between about 100 nm to about 10 micrometers (μm). Accordingly, an average particle size of the nanoparticle may be less than or equal to about 10 μm, optionally less than or equal to about 9 μm, optionally less than or equal to about 8 μm, optionally less than or equal to about 7 μm, optionally less than or equal to about 6 μm, optionally less than or equal to about 5 μm, optionally less than or equal to about 4 μm, optionally less than or equal to about 3 μm, optionally less than or equal to about 2 μm, optionally less than or equal to about 1 μm, optionally less than or equal to about 900 nm, optionally less than or equal to about 800 nm, optionally less than or equal to about 700 nm, optionally less than or equal to about 600 nm, and in certain variations, optionally less than or equal to about 500 nm.

The nanoparticle may have a round shape (e.g., a sphere or spheroid shape) or may have a variety of other shapes, such as discs, platelets, rods, and the like.

In certain variations, the nanoparticles of the present disclosure may be therapeutic. Therapeutic nanoparticles may comprise at least one oral care active ingredient. The nanoparticles of the present disclosure having such a therapeutic oral care active ingredient permit the active ingredient to be delivered to surface of teeth in the oral cavity (e.g., to a region adjacent to an active carious lesion in a tooth) to provide therapeutic benefits. An oral care active ingredient may be used for the prevention or treatment of a condition or disorder of hard or soft tissue of the oral cavity, including but not limited to oral cancer and dry mouth, the prevention or treatment of a physiological disorder or condition, or may provide a cosmetic benefit. Optional oral care active ingredients include an anticaries agent, a remineralizing agent, an antibacterial agent, an anticalculus agent, a tartar control agent, a tooth desensitizer, and combinations thereof, by way of non-limiting example. While general attributes and properties of each of the above categories of actives may differ, there may some common attributes and any given material may serve multiple purposes within two or more of such categories of actives.

In certain variations, the oral care active ingredient comprises an anticaries agent, a remineralizing agent, an antibacterial agent, an anticalculus agent, and combinations thereof. In certain variations, the nanoparticles may be used to reduce or inhibit early enamel lesions or microcavities; reduce or inhibit formation of dental caries or cavities; reduce or inhibit demineralization and promote remineralization of the tooth; protect teeth from cariogenic bacteria; inhibit microbial biofilm formation on the tooth or in the oral cavity; and/or reduce levels of acid-producing bacteria in the oral cavity. Oral care actives that are useful herein are optionally present in the compositions of the present invention in safe and effective amounts. In certain aspects, the nanoparticle comprises an oral care active ingredient of about 0.1% to less than or equal to about 75% by weight after incorporation into the nanoparticle, and optionally about 0.1% to less than or equal to about 60% by weight of the oral care active ingredient after incorporation into the nanoparticle.

In certain variations, the oral care active ingredient comprises an anti-caries active ingredient that is selected from the group consisting of: a fluoride-containing compound, a calcium-containing compound, a phosphate-containing compound, a calcium and phosphate-containing compound, and combinations thereof.

For example, the oral care active ingredient may be an anti-caries fluoride-containing component that provides fluorine ions in the oral cavity. In certain aspects, the fluoride containing active compound may be a fluoride salt, such as calcium fluoride, sodium fluoride, magnesium fluoride, strontium fluoride, stannous fluoride, and combinations thereof. In other variations, the fluoride-containing component may be selected from the group consisting of: sodium monofluorophosphate, amine fluoride, fluorohydroxyapatite, silver fluoride dehydrate, difluorosilane, combinations and equivalents thereof.

The anticaries active ingredient may be present in the nanoparticle at greater than or equal to about 20 weight %. In the case of fluoride-containing active ingredients, these may be present at greater than or equal to about 20% to less than or equal to about 75% by weight after incorporation into the nanoparticle, optionally greater than or equal to about 25% to less than or equal to about 70% by weight, optionally greater than or equal to about 30% to less than or equal to about 65% by weight, optionally greater than or equal to about 35% to less than or equal to about 60% by weight, and in certain variations, optionally greater than or equal to about 45% to less than or equal to about 60% by weight after incorporation into the nanoparticle.

Advantageously, as will be described further below, the nanoparticles provide the advantage of localized delivery of fluoride ions at the cariogenic biofilm on the tooth surface, while minimizing the adverse effects typically associated with fluoride ions. With all fluoride uses, there is a risk of toxicity-acute or chronic (fluorosis). For example, children in particular have low tolerance for high levels of fluoride-releasing anticaries agents. In accordance with certain principles of the present disclosure, substantivity of fluoride-releasing agents is increased, so that the concentration/amount or frequency of use of a fluoride product can be reduced. As such, there is the ability to reduce chronic toxicity (fluorosis) with the use of such nanoparticles, without diminishing the anticaries effect.

As previously noted, due to low efficacy and retention, conventional use of fluoride delivers high concentrations of fluoride, only a small concentration of which is effective in providing the desired anticaries effect. For the nanoparticles prepared in accordance with the present disclosure, the fluoride-releasing compound has high retention in the mouth and on the tooth surface and thus concentrated efficacy at the locus of caries where cariogenic bacteria exist to provide far greater efficacy. For example, where the anticaries agent is calcium fluoride (CaF₂), it is estimated that including about 45 to 50 weight % of calcium fluoride provides about 24 weight % of fluoride ions at the tooth surface when the nanoparticle is associated with the biofilm matrix. In certain aspects, calcium fluoride is believed to be particularly advantageous, because it has a lower solubility and thus lower bioavailability than other fluoride-releasing compounds in the oral cavity and thus can provide a longer, sustained release profile when incorporated into nanoparticles delivered into the mouth.

When an ingredient is present as an anticaries ingredient, it is desirably present at high concentrations in the nanoparticle in accordance with certain aspects of the present disclosure, for example, at greater than or equal to about 20 weight %. However, the nanoparticles may include a first oral care active ingredient and an oral care active ingredient distinct from the first oral care active. This may involve incorporating two or more distinct anticaries agents that are cumulatively present at greater than or equal to about 20 weight % of the particle or may involve incorporating an anticaries oral care active ingredient with a second oral care active ingredient that has a distinct therapeutic role (e.g., remineralizing) and thus may be present at lower concentrations.

In certain variations, an anticaries ingredient in the nanoparticle may be a fluoride salt as noted above. A number of different fluoride salts are used in dental products, such as sodium fluoride, stannous fluoride, and calcium fluoride. The sodium fluoride is highly soluble in aqueous solutions, for example, it dissolves in water up to a concentration of 18,000 ppm F (parts per million of fluoride). Stannous fluoride is similarly highly soluble, it dissolves in water up to the concentration of 42,000 ppm F. However, calcium fluoride is usually not a fluoride source for dental products because of its low solubility (for example, releasing fluoride over weeks). Because of its low solubility, it is challenging to produce homogeneous formulations of calcium fluoride. For example, in a solution containing 226 ppm F as CaF₂ (an over-the-counter concentration of fluoride rinses), precipitation of CaF₂ promptly occurs. Thus, in the liquid product, an upper part of the suspension will have much lower-than-expected concentrations, while a lower part will have much higher-than-expected concentrations. This can be particularly problematic considering that if ingested at high concentrations, fluoride is toxic (the limit for over-the-counter concentration in the United States is 226 ppm F).

In certain aspects, the fluoride-based compositions provided by the present disclosure can stabilize fluoride salts, including calcium fluoride, in aqueous compositions. In certain variations, as will be described further below, a nanoparticle prepared in accordance with certain aspects of the present disclosure comprising an active ingredient can modulate release of the ingredient. For example, where the active ingredient comprises a fluoride salt, the nanoparticles provide the ability to modulate release, for example, by providing an increased release rate/relatively quick release of fluoride from a salt having low solubility in aqueous solutions or to reduce a release/relative slow release of fluoride from a nanoparticle comprising a fluoride salt having high solubility in aqueous solutions.

In certain aspects, the fluoride-based compositions provided by the present disclosure can stabilize fluoride salts, including calcium fluoride, which has low solubility in aqueous solutions. Thus, calcium fluoride incorporated into a nanoparticle prepared in accordance with certain aspects of the present disclosure can provide a slow/sustained release of fluoride ions in a homogeneous suspension in certain variations. For example, a chitosan-calcium fluoride nanoparticle (“ChitCaF₂np”) can serve as a slow fluoride release reservoir. A ChitCaF₂np suspension containing 725 ppm F is stable, without the formation of precipitates, for days. After 7 days, only approximately 20% of the fluoride in the nanoparticle is released (e.g., at neutral pH). This increases to an approximate 30% of fluoride released at 21 days. As such, the nanoparticles comprising calcium fluoride (e.g., ChitCaF₂np) in suspension can serve as a slow-release reservoir for fluoride ions. In contrast, for comparison, a solution having a concentration of 725 ppm CaF₂ forms insoluble precipitates (of CaF₂) within 15 minutes of preparation. Thus, calcium fluoride-based nanoparticles prepared in accordance with certain aspects of the present disclosure are demonstrated to stabilize CaF₂ (that is poorly soluble) in an aqueous medium, advantageously have a controlled release of fluoride over time, are retained in the dental biofilm, and demonstrate an anticaries effect of fluoride using a biofilm model.

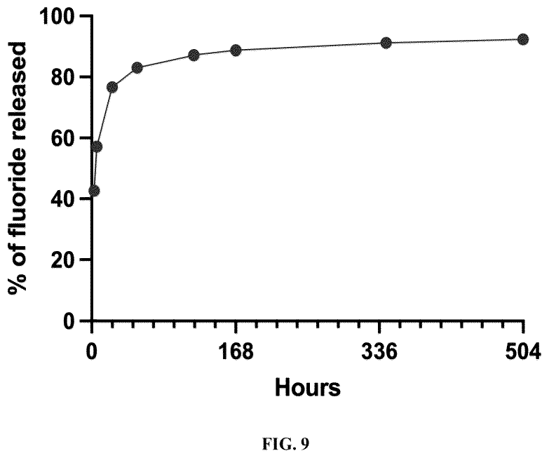

Similarly, the present disclosure contemplates a nanoparticle comprising an anticaries agent comprising stannous fluoride (SnF₂), for example, a chitosan-stannous fluoride nanoparticle (“ChitSnF₂np”) having controlled release of fluoride ions in an aqueous medium. The ChitSnF₂np can stabilize stannous fluoride (or other highly soluble fluoride salts, like sodium fluoride) as a quick fluoride release nanoparticle. A conventional aqueous-based solution of SnF₂ has all fluoride dissociated as fluoride ions (F⁻). This does not facilitate the retention of the fluoride in the mouth. However, a ChitSnF₂np prepared in accordance with the present disclosure is stable in an aqueous solution/medium, for example, with less than 25% of the formulation being released as free as fluoride ions (F⁻) in the first 25 days after the composition is prepared. The formulation releases fluoride over time at a neutral pH providing a relatively slow-release composition. For example, in one variation, approximately 80% of the fluoride present in the nanoparticle is released within 24 hours. See FIG. 9 and Table 1 that shows total and soluble fluoride (as F⁻ ions) in an aqueous suspension of the ChitSnF₂np prepared in accordance with certain aspects of the present disclosure from up to 25 days.

TABLE 1

| Days After | | Soluble F | % of Total That |
|---|---|---|---|
| Preparation | Total F (ppm) | (ppm F) | Is Soluble |
| 0 | 134.5 ± 1.2 | 20.4 ± 0.16 | 15.2% |
| 2 | 134.4 ± 0.8 | 23.6 ± 0.05 | 17.6% |
| 25 | 136.3 ± 3.4 | 31.3 ± 0.7 | 23% |

Thus, nanoparticles comprising stannous fluoride (SnF₂) particles are stable in a concentrated suspension, for example, maintaining less than 25% free fluoride even after 25 days. However, such nanoparticles can quickly release fluoride, for example, once they are diluted in a greater volume of liquid, they quickly release the fluoride. In comparison, a solution of SnF₂ in water would have all the approximately 135 ppm of fluoride ions free (SnF₂ is a very soluble fluoride salt). Thus, the nanoparticles prepared in accordance with certain aspects of the present disclosure can keep fluoride sequestered (Table 1), therefore slowing the dissolution of a very soluble fluoride salt, while enabling a fast release rate of fluoride once dissolved in a greater volume of solution (FIG. 9), while the nanoparticles also have properties permitting them to attach to surfaces in the oral cavity (especially the dental biofilm). In FIG. 9, fast-release nanoparticles comprising stannous fluoride (ChitSnF₂NP) can keep fluoride sequestered in the nanoparticle, while providing the ability to quickly release fluoride in an aqueous solution.

In certain aspects, an oral care composition prepared in accordance with the present disclosure is a mouth rinse that comprises an aqueous medium and a nanoparticle that comprises an anticaries active ingredient that may be calcium fluoride or stannous fluoride. With these nanoparticles, stable CaF₂ rinses can be formulated, and as well stable SnF₂ rinses, where the nanoparticles comprising the anticaries active agents can then bind to the biofilm/oral cavity. This provides an ability to sequester fluoride over time, while also providing an ability to modulate the fast release of fluoride.

Additionally, in certain variations, the oral care active ingredient comprises a calcium-containing component that provides calcium ions in the oral cavity for remineralizing the tooth. The calcium-containing active ingredient component may be present at greater than or equal to about 20% to less than or equal to about 60% by weight after incorporation into the nanoparticle. The calcium-containing active ingredient may be calcium lactate.

As noted above, active ingredients like calcium-phosphate compounds are anticaries agents. Oral care active ingredient comprising a calcium and phosphate-containing component can remineralize the tooth. The calcium and phosphate-containing component optionally comprises calcium glycerophosphate, dicalcium phosphate, tricalcium phosphate, calcium sodium phosphosilicate, or combinations and equivalents thereof. In certain variations, the calcium-phosphate compounds may be present in the nanoparticle at greater than or equal to about 20% to less than or equal to about 75% by weight, optionally at greater than or equal to about 20% to less than or equal to about 60% by weight, and optionally greater than or equal to about 45% to less than or equal to about 60% by weight, after incorporation into the nanoparticle after incorporation into the nanoparticle after incorporation into the nanoparticle.

In other aspects, the nanoparticle may comprise an oral care active ingredient selected from the group consisting of: amine fluoride, casein phosphopeptide, phosphoprotein, and equivalents and combinations thereof. Again, the oral care active ingredient may be present at greater than or equal to about 20% to less than or equal to about 75% by weight, optionally at greater than or equal to about 20% to less than or equal to about 60% by weight, after incorporation into the nanoparticle after incorporation into the nanoparticle.

The nanoparticles of the present disclosure also include a biofilm matrix-degrading enzyme. As noted above, the biofilm matrix may be an exopolysaccharide (EPS)-rich dental biofilm. Such an enzyme may be selected from the group consisting of: dextranase, mutanase, nucB, and combinations thereof. Dextranase and mutanase are both EPS-degrading enzymes. As discussed above, the matrix-degrading enzyme(s) facilitate the penetration of the nanoparticle and its components into biofilms on the surface of the tooth. The matrix-degrading enzymes may disintegrate parts of the biofilms, which is believed to create pores or openings, which receive and retain the nanoparticles. For example, dextranase appears to hydrolyze a 1 to 6 glycosidic linkages of biofilm polysaccharides to facilitate penetration of the nanoparticles deeper into the biofilm. Dextranase is typically produced by dental biofilm bacteria, so is commonly found in the natural oral environment. nucB is a DNAse. As noted above, biofilms contain extracellular DNA (eDNA) from dead microorganisms, so eDNA plays a role in biofilm aggregation and nucB can degrade such eDNA. The biofilm matrix-degrading enzyme may be present in the nanoparticle at greater than or equal to about 1 weight %. For example, the biofilm matrix-degrading enzyme may be present at optionally greater than or equal to about 1% to less than or equal to about 15% by weight after incorporation into the nanoparticle, optionally greater than or equal to about 2% to less than or equal to about 25% by weight.

In certain variations, the nanoparticle may be a multiphasic nanoparticle that comprises multiple compositionally distinct compartments. Each compartment may thus comprise distinct material compositions. Multiphasic nanoparticles may have a variety of shapes and may comprise two, three, or more distinct compartments. In certain variations, a first compartment may have compositional differences in the polymer or other ingredients like the matrix-degrading enzyme, as compared to the second compartment, for example, to provide different dissolution rates in vivo or to deliver higher levels of enzyme to ensure the particle penetrates into the biofilm matrix. In certain variations, a first compartment may include one or more oral care active ingredients, while the second compartment may have one or more distinct oral care active ingredients. In certain variations, multicompartmental nanoparticles may be employed where two active ingredients, e.g., a fluoride compound and an enzyme, are incorporated in different compartments or phases of the same nanoparticle. Similarly, multicompartmental nanoparticles may be employed where the active ingredients and imaging compounds, e.g., dyes, are incorporated in different compartments of the same nanoparticle. Such multiphasic nano-components may be formed by electrified jetting of materials that comprise one or more polymers, such as that disclosed by Roh et al., “Biphasic Janus Particles With Nanoscale Anisotropy”, Nature Materials, Vol. 4, pp. 759-763 (October 2005), as well as in U.S. Pat. Nos. 7,767,017, 8,043,480, 8,187,708, and in U.S. Publication No. 2012/0045487 and PCT International Publication No. WO 06/137936, the relevant portions of which are incorporated herein by reference.

In certain aspects, the present disclosure contemplates an oral care composition for oral administration in an oral cavity of a subject. In certain aspects, the nanoparticle is delivered to the mouth to contact and bind with one or more of the following surface: oral surfaces (hard and soft tissue), teeth, biofilms (e.g., dental plaque), and anionic surfaces (e.g., enamel, dentin, carious lesions, and cavities). More specifically, in certain variations, the oral care composition may be introduced into the oral cavity of the subject, so that it contacts surfaces of teeth. In certain aspects, only a single oral care composition is delivered to the mouth in a single step while providing desired efficacy, instead of delivering multiple distinct oral care compositions sequentially. The oral care composition includes any of the nanoparticles discussed above. The nanoparticle also includes an orally acceptable carrier, meaning a material or combination of materials that are relatively safe for use within a subject while considering the risks versus benefits (e.g., that the benefits outweigh the risks). An orally acceptable carrier may thus be any carrier toxicologically suitable for use in the oral cavity. Selection of specific components of the orally acceptable carrier depend upon the form of the oral care composition, for example, whether the oral care composition is a mouth rinse, dentifrice, gel, film, adhesive strip, sponge or pellet, or the like. Such orally acceptable carriers include the usual components of dentifrices (e.g., toothpastes and tooth powders), topical gels, topical varnishes, paints, mouth rinses (e.g., such as a mouth wash, spray, or rinse), lozenges, troches, intraoral film, dissolvable adhesive strips, pellets or beads for adhering to teeth, sponges or pellets to release composition to water reservoirs, chewing gum, a chewing tablet, and the like, as are well known to those of skill in the art. In certain variations, the oral care composition facilitates extensive and comprehensive coverage of surfaces of teeth, including interproximal/interdental surfaces where caries often tends to develop.

In various aspects, the orally acceptable carrier used to prepare an oral composition may comprise a water-based phase (e.g., aqueous phase), which may include alcohols and other components. As recognized by one of skill in the art, the oral compositions may include other conventional oral care composition materials, including by way of non-limiting example, surface active agents, such as surfactants, emulsifiers, and foam modulators, abrasives, humectants, mouth feel agents, viscosity modifiers, diluents, pH modifying agents, sweetening agents, flavor agents, colorants, preservatives, and combinations thereof.

The oral care composition comprising the nanoparticles may be administered to the subject and thus introduced into the oral cavity of the subject for contacting the target oral surfaces, like surfaces of teeth. The plurality of particles selectively accumulates in the biofilm matrix adjacent to caries on the surface of a tooth. The oral care composition may include only one type of nanoparticle or alternatively multiple distinct types of nanoparticles, as previously discussed above. Thus, the plurality of nanoparticles includes a plurality of therapeutic nanoparticles comprising an oral care active ingredient.

In certain aspects, the present disclosure provides nanoparticle compositions and metabolites that are non-toxic and resorbable in contrast to certain synthetic polymers that can potentially cause side effects and toxicity when used in medical diagnostic applications. In various aspects, the nanoparticles have an advantageous size for embedding and/or penetrating a biofilm matrix, such as exopolysaccharide (EPS)-rich dental biofilms being of a particle size that permits entry into cavities and lesions in tooth enamel. The nanoparticles according to certain variations of the present disclosure are easy to functionalize, allowing for the attachment of various fluorescent or optical dyes or imaging agents, protective coatings, and control over particle charge. For example, a reactive functional group may be a carboxyl group on the polymer that reacts with an amine on an imaging particle (e.g., an amine-functionalized imaging agent). Another variation may include reacting an alkyne functional group on the polymer by copper-click chemistry on the corresponding imaging agent. Other variations include use of carbodiimides (EDC) that cause direct conjugation of carboxyls (—COOH) to primary amines (—NH₂) without becoming part of the final crosslink (amide bond) between target molecules. N-hydroxysuccinimide (NHS) or its water-soluble analog (Sulfo-NHS) can be included in EDC coupling processes to enhance bonding. Other examples of conjugation chemistries include the reaction of azides with phosphines, thiols with maleimide or vinyl groups, or photoinduced cross-linking of photoreactive groups, such as benzophenone. Furthermore, certain variations of the present disclosure provide nanoparticles that can be manufactured on an industrial scale with high production rates for relatively low cost, compared by many micro and nanoparticle systems that are limited by the ability to scale up production.

In various aspects, the nanoparticles and oral compositions contemplated by the present teachings can be used for one or more of the following applications: administration for treatment or prevention of cavities in a dental office by a clinician (e.g., dentist, dental assistant, or hygienist), home treatment or prevention of dental caries or cavities. In certain variations, the method may include preventing or treating caries in a tooth in an oral cavity of a subject, including introducing a plurality of nanoparticles into the oral cavity of the subject so that the plurality of the nanoparticles selectively accumulates within a biofilm matrix associated with a surface of the tooth in the oral cavity of the subject. Each nanoparticle of the plurality of nanoparticles comprises a biocompatible and biodegradable hydrophilic polymer, a biofilm matrix-degrading enzyme, and an anticaries active ingredient present in the nanoparticle at greater than or equal to about 20% by weight, the nanoparticle having a zeta potential between about −10 mV to about +10 mV at a pH of 7, as described previously above. Treatment with such nanoparticles may provide the ability to revert cariogenic biofilm dysbiosis and reduce caries progression in a patient.

In certain variations, the anticaries active ingredient comprises a fluoride salt. As noted above, certain fluoride salts that have lower solubility in aqueous environments may be selected to provide longer release profiles. For example, calcium fluoride (CaF₂) has a relatively low solubility as compared to more commonly used sodium fluoride (NaF), although both would be suitable for use as a fluoride-releasing active compound in the nanoparticle. Further, certain fluoride salts that have high solubility in aqueous environments and may be tailored to provide a quick release rate and release profile (albeit being longer than the highly soluble salt alone). For example, stannous fluoride (SnF₂) has a relatively high solubility and is suitable for use as a fluoride-releasing active compound in the nanoparticle. In certain variations, the nanoparticle may be configured to deliver greater than or equal to about 1 ppm to less than or equal to about 25 ppm of fluoride ions, optionally greater than or equal to about 1 ppm to less than or equal to about 20 ppm of fluoride ions, optionally greater than or equal to about 1 ppm to less than or equal to about 15 ppm of fluoride ions, optionally greater than or equal to about 1 ppm to less than or equal to about 10 ppm of fluoride ions to the biofilm matrix associated with the surface of the tooth. In certain variations, the nanoparticle may be configured to deliver greater than or equal to about 1 ppm to less than or equal to about 25 ppm of fluoride ions for greater than or equal to about 8 hours to the biofilm matrix associated with the surface of the tooth. In certain other variations, the nanoparticle may be configured to deliver greater than or equal to about 20% to less than or equal to about 30% of the total fluoride ions in the nanoparticle over a duration of greater than or equal to about 7 days, optionally greater than or equal to about 21 days (for example, when the active ingredient comprises calcium fluoride). In yet other variations, the nanoparticle may be configured to deliver greater than or equal to about 20% to less than or equal to about 80% of the total fluoride ions in the nanoparticle over a duration of greater than or equal to about 1 day, optionally greater than or equal to about 25 days (for example, when the active ingredient comprises stannous fluoride).

The nanoparticle may be configured to deliver fluoride ions at these levels (or other active ingredients at clinically efficacious levels) to the biofilm matrix/locus of caries at the tooth surface for any of the durations described above corresponding to a given lifetime of the nanoparticle in the oral cavity as it is in contact with saliva and an aqueous environment. For example, in certain aspects, the anticaries active ingredient has substantivity on the biofilm matrix associated with the surface of the tooth for greater than or equal to about 8 hours to less than or equal to about 1 week, optionally greater than or equal to about 1 day to less than or equal to about 7 days, and optionally greater than or equal to about 2 days to less than or equal to about 7 days, by way of non-limiting example.

In various aspects, the methods of the present disclosure provide one or more of the following benefits: enhancement of an intraoral concentration of fluoride, enhancement of fluoride concentration in dental biofilm, enhancement of fluoride concentration on the surface of oral mucosa, enhancement of fluoride concentration on the surface of teeth, enhancement of fluoride concentration in saliva, reduction of tooth (e.g., enamel, dentin) demineralization, enhancement of tooth (e.g., enamel, dentin) remineralization, prevention of dental caries, prevention of coronal caries, prevention of root caries, prevention of caries in high caries-risk patients, prevention of caries in hyposalivation patients, prevention of early childhood caries, treatment of dental caries, and treatment of coronal caries, prevention of biofilm-mediated diseases (e.g., gingivitis, periodontitis, denture stomatitis, oral candidiasis), inhibition of acid production by dental biofilm bacteria, inhibition of the metabolism of dental biofilm bacteria, prevention of dental fluorosis, prevention of skeletal fluorosis, prevention and/or mitigation of toxic effects of fluorides (e.g., reduction of systemic fluoride absorption).

In one variation, the present disclosure contemplates nanoparticles having a biocompatible and biodegradable hydrophilic polymer comprising chitosan having a molecular weight (number weight) of greater than or equal to about 30 kDa to less than or equal to about 120 kDa. In other variations, the nanoparticles may comprise chitosan having a molecular weight (number weight) of greater than or equal to about 310 kDa to less than or equal to about 375 kDa. The nanoparticle may further include calcium fluoride (CaF₂) as the fluoride-releasing anticaries active ingredient present at greater than or equal to about 45% by weight after incorporation into the nanoparticle to less than or equal to about 60% by weight. The matrix-degrading enzyme comprises dextranase.

In another variation, the present disclosure contemplates nanoparticles having a biocompatible and biodegradable hydrophilic polymer comprising chitosan having a molecular weight (number weight) of greater than or equal to about 30 kDa to less than or equal to about 120 kDa. In other variations, the nanoparticles may comprise chitosan having a molecular weight (number weight) of greater than or equal to about 310 kDa to less than or equal to about 375 kDa. The nanoparticle may further include stannous fluoride (SnF₂) as the fluoride-releasing anticaries active ingredient present at greater than or equal to about 45% by weight after incorporation into the nanoparticle to less than or equal to about 60% by weight. The matrix-degrading enzyme comprises dextranase.

Various embodiments of the inventive technology can be further understood by the specific examples contained herein. Specific Examples are provided for illustrative purposes of how to make and use the compositions, devices, and methods according to the present teachings and, unless explicitly stated otherwise, are not intended to be a representation that given embodiments of this invention have, or have not, been made or tested.

### EXAMPLES

In early studies, it was determined that a fluoride treatment able to form calcium fluoride can increase fluoride concentration in the dental biofilm more than 20 times, and consequently improve the effect of a fluoride-only treatment used at the same concentration. Therefore, treatment is provided to target the penetration and retention of calcium fluoride in dental biofilm, with an improved fluoride-releasing capacity. The treatment comprises a chitosan-calcium fluoride nanoparticle (Chit-CaF₂np, a fluoride-releasing cationic particle) combined with dextranase (Dex, an enzyme able to degrade the extracellular polysaccharide matrix of cariogenic dental biofilms). It is believed that with the aid of the dextranase, Chit-CaF₂NP penetrates the cariogenic biofilm, binds to negatively-charged biofilm components, and releases fluoride at levels high enough to improve the physicochemical effect of fluoride (needed for the control of root caries) as well as inhibits acid production by the biofilm (reducing the predominance of acid-producing species, e.g., biofilm dysbiosis). This improved anticaries approach is believed to provide an ability to reduce the cariogenicity of dental biofilm and control root caries. This contribution is significant in addressing the need to control rampant caries progression in high caries-risk groups, such as older adults suffering from hyposalivation.

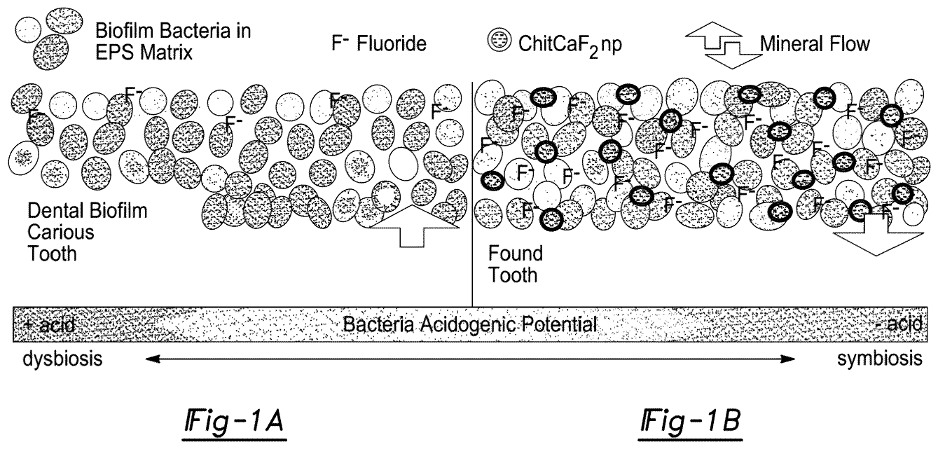

FIG. 1 shows a schematic of a proposed effect of the Chit-CaF₂+Dex nanoparticles prepared in accordance with certain aspects of the present disclosure. FIG. 1 shows bacteria acidogenic potential ranging from dysbiosis on the left-side to symbiosis on the right-side. On left-side (A) of FIG. 1, fluoride from currently available treatments has limited capacity to bind to dental biofilm, on right-side (B) in FIG. 1, enhanced availability of fluoride in dental biofilm by the present methods is shown. The dextranase component (not shown) facilitates penetration of chitosan-CaF₂ nanoparticles (Chit-CaF₂NP) into the biofilm, where they will be retained due to their positive nature for continuous fluoride release. Fluoride will reduce bacterial acid production and potentiate mineral gain by the tooth (reducing demineralization and enhancing remineralization). In this manner, it is believed that the nanoparticles of the present teachings can improve use of the best anticaries agent (e.g., fluoride), without the need for using increased concentrations, by retaining it at a proper location in the mouth for a greater effect.

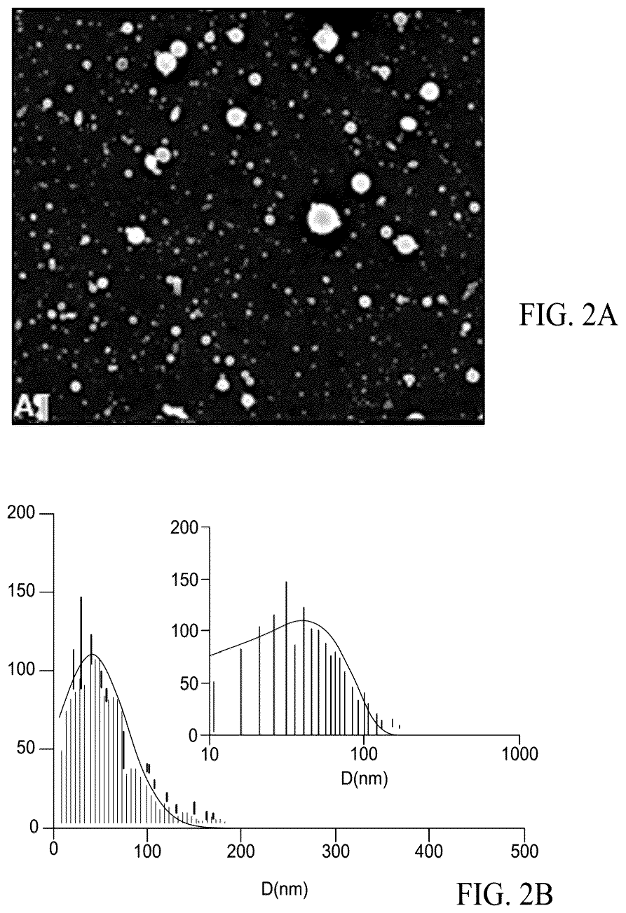

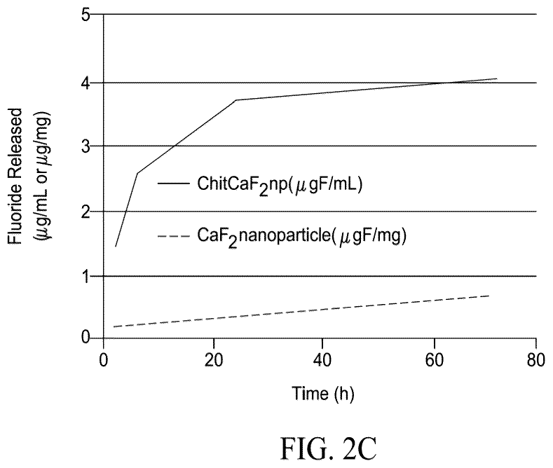

The present disclosure provides in certain aspects, a one-step treatment to enhance penetration and retention of CaF₂ in dental biofilms for an improved anticaries effect by use of oral care compositions having nanoparticles comprising CaF₂. The chitosan-CaF₂ nanoparticles (Chit-CaF₂NP) are produced via electrodynamic co-jetting, as discussed above, resulting in mild positively charged, fluoride-releasing nanoparticles (preliminary data showing a zeta potential of +3.91 mV, FIGS. 2A-2C). Chitosan was chosen as the platform to deliver CaF₂ due to its mildly cationic nature (improving biofilm retention) as well as being biocompatible. Moreover, the rate of fluoride release from the Chit-CaF₂NP is accelerated when compared with CaF₂ nanoparticles alone (FIG. 2C), demonstrating its capacity to enrich the biofilm with free fluoride. In particular, although CaF₂ is considered a slow-release fluoride source due to its low solubility (KₛₚCaF₂=3.9×10₋₁₁), data show the nanoparticle releases fluoride, and at a higher dissolution rate, than CaF₂ nanoparticles (FIG. 2C), which is desirable for a treatment intended to affect biofilm dysbiosis. Stated in another way, FIG. 2C shows that CaF₂ loaded in a nanoparticle is more soluble than a nano CaF₂ nanocrystal.

Moreover, a high number of bound particles will potentiate fluoride release and elevate free fluoride levels in the fluid of the biofilm.

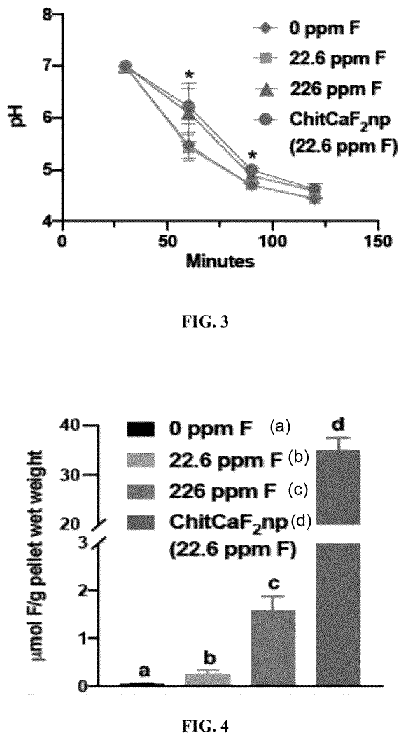

FIG. 3. shows an experiment where there is an inhibition of acid production by a cariogenic bacterium (Streptococcus mutans), which is similar to treating these microorganisms with 10 times higher fluoride concentration (22.6 ppm in the nanoparticles formed in accordance with the present disclosure versus 226 ppm of free fluoride from sodium fluoride particles). In FIG. 3, inhibition of acidogenicity by the ChitCaF2NPs are shown. First, S. mutans were treated with solutions with increasing fluoride concentrations (sodium fluoride (NaF)) or with a suspension of the ChitCaF₂NP. After treatment removal and washing, cells were resuspended in TSB medium containing 1% glucose, and pH was assessed over time. * ChitCaF₂NP prepared in accordance with certain variations of the present disclosure differ significantly from the 22.6 ppm F group at 60 and 90 min (p<0.05). This experiment shows that there is an inhibition of acid production by a cariogenic bacterium (Streptococcus mutans), which is similar to treating these microorganisms with 10 times higher fluoride concentration (22.6 ppm in the nanoparticle×226 ppm of free fluoride).

FIG. 4 shows binding of ChitCaF₂NP to S. mutans. S. mutans cells were treated with NaF solutions (0, 22.6, or 226 ppm F) or with a ChitCaF₂NP suspension prepared in accordance with certain aspects of the present disclosure (22.6 ppm F). Unbound particles separated from pellets by centrifugation at 500×g; control tubes containing only ChitCaF₂NP were used to discount fluoride coming from unbound precipitated particles (35.7±1.8%). Total fluoride in the bacterial pellets was extracted using 0.5 M HCl. Different letters show differences between treatments at p<0.0001. Average±SD, n=4. From this experiment, it seems that the particles actually bind to the bacterial cells; after removing unbound particles, concentration in bacterial pellets is still much higher than fluoride alone.

FIG. 5 shows chitosan-calcium fluoride nanoparticles (ChitCaF₂np) capable of binding to the dental biofilm, facilitated by a dextranase (Dex) treatment to increase their penetration into the biofilm matrix. Preliminary data indicates that this treatment increases the retention of fluoride in the dental biofilm around 100 times (FIG. 5). Notably, the effect of dextranase is significant only for the ChitCaF₂ nanoparticles. It is hypothesized that treatment with nanoparticles prepared in accordance with certain aspects of the present disclosure that include an enzyme, like dextranase, which degrades the exopolysaccharide (EPS) matrix of caries-associated biofilms facilitates that penetration and retention of the ChitCaF₂ nanoparticles into the biofilm, which may further improve their pH neutralizing potential. The preliminary data in FIG. 5 thus suggests that there is a dose response effect of dextranase on ChitCaF₂ nanoparticles concentration in a cariogenic biofilm. With such an increased retention, lower fluoride concentrations can result in an enhanced anticaries effect. Therefore, this technology provides the ability to improve the effectiveness of fluoride products for root caries control and mitigate the concerns associated with fluoride toxicity, allowing the development of products with greater effect and lower fluoride concentrations.

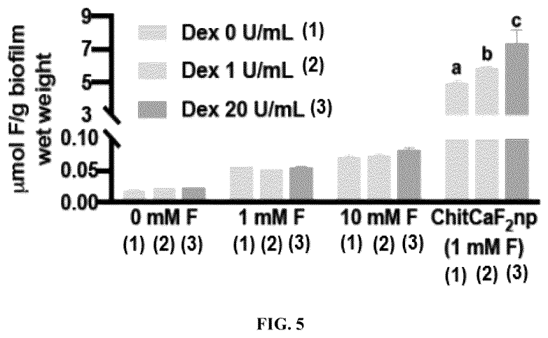

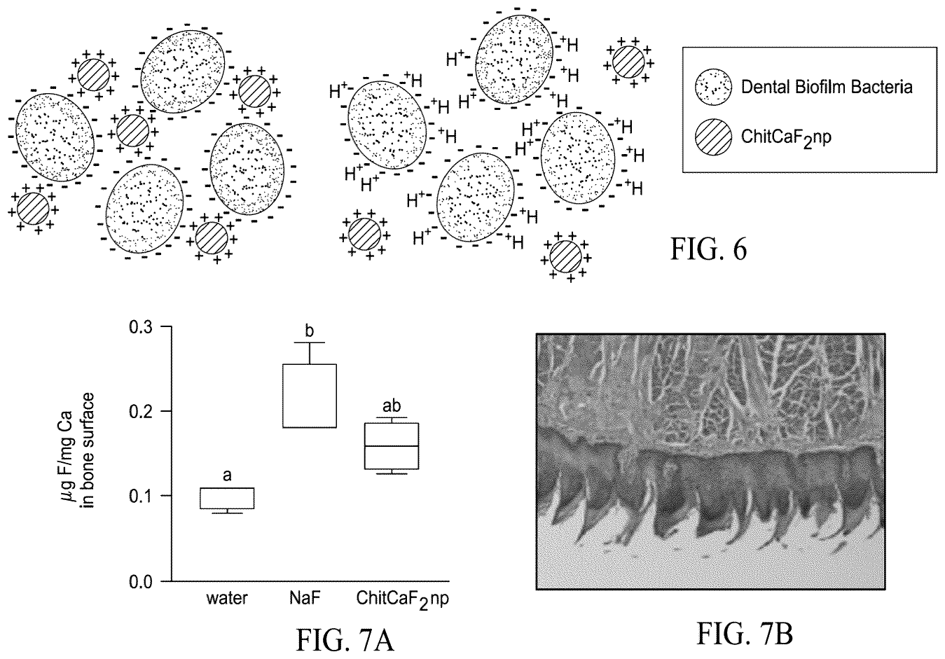

FIG. 6 shows a proposed binding mechanism for nanoparticles prepared in accordance with certain variations of the present disclosure (ChitCaF₂NP). The figure shows protonation of negatively charged groups and release of the Chit-CaF₂np during a pH drop.

FIGS. 7A-7B show reduced fluoride bioavailability and enhanced safety of nanoparticles prepared in accordance with certain variations of the present disclosure (ChitCaF₂NP). In FIG. 7A, fluoride in the femur of rats treated with 226 ppm F (NaF or ChitCaF₂NP) or deionized water is shown. Boxplot, different letters show significant differences at p<0.005. FIG. 7B shows histopathological analysis of the lateral border of the tongue in a ChitCaF₂NP-treated animal (H&E, 10×). Methods: 21-days-old Sprague-Dawley rats (n=4/group) treated intraorally with 0.1 mL of the treatments using a brush, daily for 3 weeks. Fluoride extracted from bone by a 1-min acid biopsy. The ChitCaF₂np+Dex therapy is believed to be biocompatible and not cause any harm to oral soft tissues or organs. Moreover, the calcium component of the treatment may reduce fluoride absorption and bioavailability. In this animal study (rat study, in vivo), animals treated with the ChitCaF₂ nanoparticles had less circulating fluoride (and therefore less fluoride incorporated into the bone) than those treated with the same concentration of free fluoride (from NaF). This is believed to point to the present technology being a safer treatment method than conventional fluoride treatments.

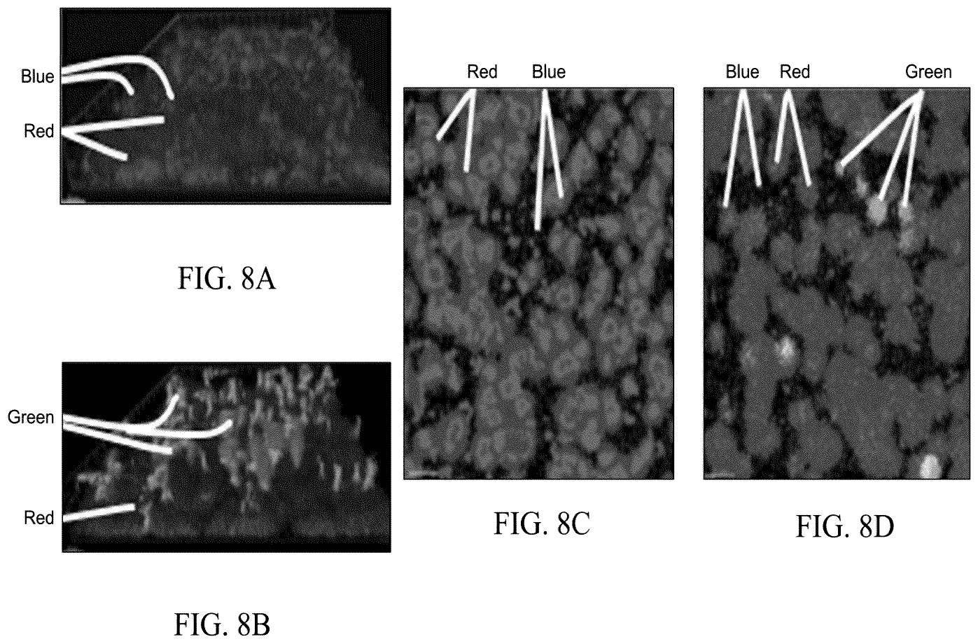

An experiment supporting the use of dextranase (Dex) or other EPS degrading enzymes to create pores in the biofilm or EPS matrix into which nanospheres can penetrate is shown in FIGS. 8A-8D. In this proof of concept experiment, treatments were done separately, first with application of dextranase, then with commercially available nanospheres having a diameter of about 200 nm as a proxy. Biofilms treated with placebo (FIGS. 8A, 8C) or dextranase (FIGS. 8B, 8D) followed by the addition of nanospheres. Red=S. mutans 3209/pVMCherry; Blue=EPS; Green=fluorescent nanospheres. In FIG. 8B, large accumulations of nanospheres are clearly visible penetrating into biofilm pores. FIGS. 8C and 8D are each from a confocal stack where each image is located deep on the biofilm (approximately 2.5 μm above the bottom). In FIG. 8D, many spheres are in intimate contact with and within the biofilms (the green channel shows 14.8 times more pixels in D than in C (Image J)). Methods: S. mutans biofilms grown in BHI broth containing 1% of sucrose and 1 μM Alexa Fluor 647 dextran conjugate for 24 h. After 1 hour of treatment with dextranase (D5884, Sigma) or placebo (0.1 M sodium acetate, pH 5.5) and washing, a 0.005% fluorescent, cationic nanosphere suspension (200 nm in diameter, F8764, Fisher) was applied as a proxy for nanoparticles formed in accordance with certain aspects of the present teachings. Images captured after approximately 2 min using a Leica TCS SPE confocal laser scanning microscope.

The nanoparticles are small in diameter (e.g., 200 nm) and thus can penetrate into the biofilm (see FIG. 8C). Penetration of such nanoparticles is much improved by the present of dextrananse (comparing FIGS. 8C and 8D, showing the pores created in the biofilm). The “pores” or openings in the biofilm created by dextranase are shown in FIG. 8B, estimated to be an average of about 10 micrometers in diameter, which is much larger than pores formed in enamel.

In vitro biofilm models can be successfully used to test the anticaries effect of anticaries formulations prepared in accordance with certain aspects of the present disclosure. In the models used in this example, one or more bacterial species are grown on the surface of tooth slabs and fed with sugar to promote the production of acids to demineralize tooth minerals. Anticaries treatments are applied 1-2 times/day.

On Day 1 of a biofilm experiment, tooth enamel slabs, with known baseline surface hardness, are mounted on the lid of a 24 well plate and sterilized. A Streptococcus mutans culture is grown overnight and an aliquot is inoculated in fresh culture media, dispensed in a 24 well plate (2 mL/well). The slabs are placed in contact with the culture media for active bacterial adhesion (slabs are suspended upside down) for the next 8 hours.

During the experiment, the plate is maintained at 37° C., 5% CO₂ environment to simulate intraoral conditions. On Days 2-4, in the morning and at the end of the day, the slabs are rinsed in saline solution (3 washes), treated for 1 minute with different anticaries solutions, rinsed again in saline solution (3 washes), and transferred to a different batch of the culture media

During 8 hours in the day, the slabs are kept in culture media containing 1% sucrose (“feast” period). During 16 hours in the night, the slabs are kept in culture media containing 0.1 mM glucose (“famine” period).

On Day 5 of the biofilm experiment, after abundant formation of biofilm on the slabs, they are carefully removed from the plate and sonicated to detach the biofilm. Surface hardness of the tooth slabs is determined again to calculate the percentage of surface hardness loss (to indicate demineralization).

The biofilm is analyzed for fluoride concentration.

Using this biofilm model, the effect of substituting 10% of the fluoride in a sodium fluoride solution with chitosan-calcium fluoride nanoparticles (ChitCaF₂np) prepared in accordance with certain aspects of the present disclosure is evaluated.

The dose response effect to the fluoride was tested using fluoride concentrations of 226 ppm F (similar to fluoride concentration in other the counter (OTC) rinses), 22.6 ppm F (10 times lower than OTC), and 2,260 ppm F (10 times higher than OTC).

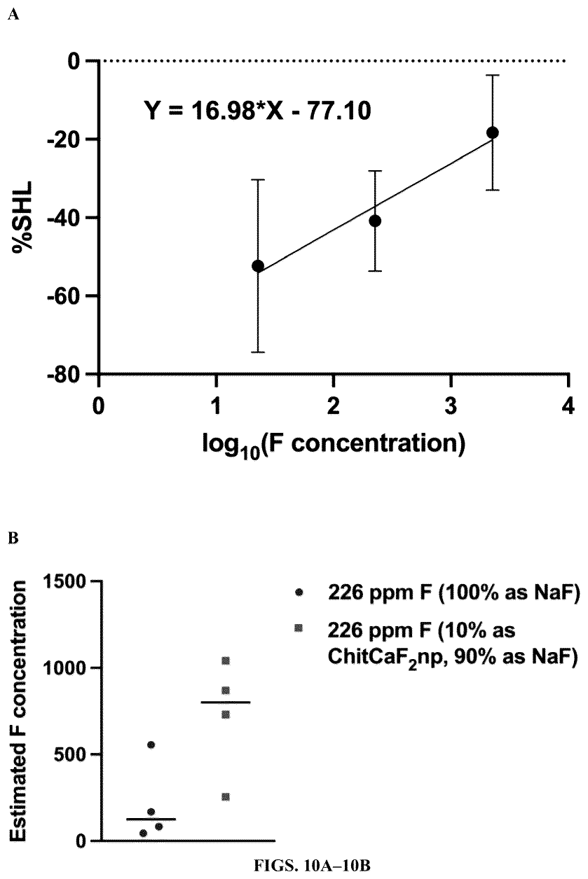

FIG. 10A shows a graph of the dose-response of the model to fluoride at increasing concentrations (22.6, 226 and 2,260 ppm F) on the % of surface hardness loss (% SHL) in the enamel slabs. There is a linear relationship between the anticaries effect and the log₁₀ of fluoride concentrations reflecting the expected anticaries effect of fluoride.

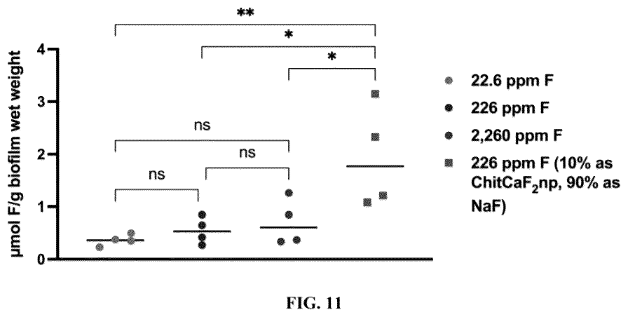

As shown in FIG. 10B, comparing a treatment with 226 ppm F where 100% is NaF and 226 ppm F having 10% as ChitCaF₂ nanoparticles and 90% as NaF, so that fluoride in the 226 ppm F formulation is substituted with only 10% of ChitCaF₂np, results in a clear improvement in the anticaries effect (estimated effect is 3 times greater). The improved anticaries effect is believed to be attributable to the fluoride concentration that is retained in the biofilm), as shown in FIG. 11. FIG. 11 shows fluoride concentration in an S. mutans biofilm 16 hours after the last treatment with solutions containing 22.6, 226 and 2,260 ppm F or 226 ppm F with 10% of fluoride in the form of nanoparticles prepared in accordance with certain variations of the present disclosure (ChitCaF₂NP) and 90% of fluoride as NaF. The group treated with 226 ppm F with 10% being in the form of ChitCaF₂ nanoparticles has significantly more fluoride than the other 3 groups (on average 2.7 times more fluoride retained in the biofilm than the group containing 10 times more fluoride).

The biofilm was collected and analyzed 16 hours after the treatments, suggesting a long-term effect which is in accordance with the slow fluoride release of the formulation.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A biofilm-targeting nanoparticle for treating a tooth in an oral cavity of a subject comprising:
a biocompatible and biodegradable hydrophilic polymer;
a matrix-degrading enzyme; and
an anticaries active ingredient present in the nanoparticle at greater than or equal to about 20% by weight, the nanoparticle having a zeta potential between about −10 mV to about +10 mV at a pH of 7.

2. The biofilm-targeting nanoparticle of claim 1, wherein the anticaries active ingredient is selected from the group consisting of: a fluoride-containing compound, a calcium-containing compound, a phosphate-containing compound, and/or a calcium and phosphate-containing compound.

3. The biofilm-targeting nanoparticle of claim 1 wherein the anticaries active ingredient is present at greater than or equal to about 25% to less than or equal to about 70% by weight of the nanoparticle.

4. The biofilm-targeting nanoparticle of claim 1, wherein the anticaries active ingredient comprises a fluoride salt.

5. The biofilm-targeting nanoparticle of claim 1, wherein the anticaries active ingredient is selected from the group consisting of: calcium fluoride, sodium fluoride, magnesium fluoride, strontium fluoride, stannous fluoride, sodium monofluorophosphate, amine fluoride, calcium-phosphate compounds, and combinations thereof.

6. The biofilm-targeting nanoparticle of claim 1, wherein the anticaries active ingredient comprises calcium fluoride and is present at greater than or equal to about 45% by weight after incorporation into the nanoparticle.

7. The biofilm-targeting nanoparticle of claim 1, wherein the anticaries active ingredient comprises stannous fluoride and is present at greater than or equal to about 45% by weight after incorporation into the nanoparticle.

8. The biofilm-targeting nanoparticle of claim 1, wherein the biocompatible and biodegradable hydrophilic polymer is selected from the group consisting of: chitosan, gelatin A, cationic dextran, pectin, dextrin, poly(amidoamine), poly(2-N,N-dimethylaminoethylmethacrylate), and combinations thereof.

9. The biofilm-targeting nanoparticle of claim 1, wherein the nanoparticle is configured to selectively accumulate within the biofilm matrix associated with the surface of the tooth for greater than or equal to about 1 day.

10. The biofilm-targeting nanoparticle of claim 1, wherein the nanoparticle has an average diameter of greater than or equal to about 10 nm to less than or equal to about 500 nm.

11. The biofilm-targeting nanoparticle of claim 1, wherein the matrix-degrading enzyme is selected from the group consisting of: dextranase, mutanase, nucB, and combinations thereof.

12. The biofilm-targeting nanoparticle of claim 1, wherein:
the biocompatible and biodegradable hydrophilic polymer is selected from the group consisting of: chitosan, gelatin A, cationic dextran, poly(amidoamine), poly(2-N,N-dimethylaminoethylmethacrylate), and combinations thereof;
the anticaries active ingredient is selected from the group consisting of: calcium fluoride, sodium fluoride, magnesium fluoride, strontium fluoride, stannous fluoride, sodium monofluorophosphate, amine fluoride, calcium-phosphate compounds, and combinations thereof; and
the matrix-degrading enzyme is selected from the group consisting of: dextranase, mutanase, nucB, and combinations thereof.

13. The biofilm-targeting nanoparticle of claim 1, wherein the biocompatible and biodegradable hydrophilic polymer comprises chitosan having a molecular weight (Mₙ) of greater than or equal to about 30 kDa to less than or equal to about 375 kDa, the anticaries active ingredient comprises calcium fluoride and is present at greater than or equal to about 45% by weight after incorporation into the nanoparticle to less than or equal to about 60% by weight, and the matrix-degrading enzyme comprises dextranase.

14. An oral care composition for treating a tooth in an oral cavity of a subject comprising:
a plurality of biofilm-targeting nanoparticles each comprising:
a biocompatible and biodegradable hydrophilic polymer;
a matrix-degrading enzyme; and
an anticaries active ingredient present in each nanoparticle at greater than or equal to about 20% by weight, each nanoparticle of the plurality of biofilm-targeting nanoparticles having a zeta potential between about −10 mV to about +10 mV at a pH of 7; and,
an orally acceptable carrier comprising water, wherein the plurality of biofilm-targeting nanoparticles is distributed in the orally acceptable carrier.

15. The oral care composition of claim 14, wherein the oral care composition is selected from the group consisting of: a mouth rinse, a dentifrice, a gel, a varnish, a paint, a lozenge, a troch, chewing gum, a chewing tablet, intraoral film, adhesive strip, a pellet or bead for adhering to teeth, a sponge or pellet, chewing gum, and a chewing tablet.

16. The oral care composition of claim 14, wherein:
the biocompatible and biodegradable hydrophilic polymer is selected from the group consisting of: chitosan, gelatin A, pectin, dextrin, cationic dextran, poly(amidoamine), poly(2-N, N-dimethylaminoethylmethacrylate), and combinations thereof;
the anticaries active ingredient is selected from the group consisting of: calcium fluoride, sodium fluoride, magnesium fluoride, strontium fluoride, stannous fluoride, sodium monofluorophosphate, amine fluoride, calcium-phosphate compounds, and combinations thereof; and
the matrix-degrading enzyme is selected from the group consisting of: dextranase, mutanase, and combinations thereof.

17. The oral care composition of claim 14, wherein the biocompatible and biodegradable hydrophilic polymer comprises chitosan having a molecular weight (number weight) of greater than or equal to about 30 kDa to less than or equal to about 375 kDa, the anticaries active ingredient comprises calcium fluoride and is present at greater than or equal to about 45% by weight after incorporation into the nanoparticle to less than or equal to about 60% by weight, and the matrix-degrading enzyme comprises dextranase.

18. The oral care composition of claim 14, wherein the oral care composition is a mouth rinse and the anticaries active ingredient is calcium fluoride or stannous fluoride.

19. A method of preventing or treating caries in a tooth in an oral cavity of a subject comprising:
introducing a plurality of biofilm-targeting nanoparticles into the oral cavity of the subject to associate with the tooth, wherein each biofilm-targeting nanoparticle of the plurality of biofilm-targeting nanoparticles comprises:
a biocompatible and biodegradable hydrophilic polymer;
a matrix-degrading enzyme; and
an anticaries active ingredient present in the nanoparticle at greater than or equal to about 20% by weight, the nanoparticle having a zeta potential between about −10 mV to about +10 mV at a pH of 7.

20. The method of claim 19, wherein the anticaries active ingredient comprises a fluoride salt that is configured to deliver greater than or equal to about 0.1 ppm to less than or equal to about 10 ppm of fluoride ions for greater than or equal to about 1 day to the biofilm matrix associated with the surface of the tooth.

21. The method of claim 19, wherein the anticaries active ingredient has substantivity on the biofilm matrix associated with the surface of the tooth for greater than or equal to about 1 day to less than or equal to about 1 week.

22. The method of claim 19, wherein the biocompatible and biodegradable hydrophilic polymer is selected from the group consisting of: chitosan, gelatin A, cationic dextran, poly(amidoamine), poly(2-N,N-dimethylaminoethylmethacrylate), and combinations thereof;
the anticaries active ingredient is selected from the group consisting of: calcium fluoride, sodium fluoride, magnesium fluoride, strontium fluoride, stannous fluoride, sodium monofluorophosphate, amine fluoride, calcium-phosphate compounds, and combinations thereof; and
the matrix-degrading enzyme is selected from the group consisting of: dextranase, mutanase, and combinations thereof.

23. The method of claim 19, wherein the biocompatible and biodegradable hydrophilic polymer comprises chitosan having a molecular weight (number weight) of greater than or equal to about 30 kDa to less than or equal to about 375 kDa, the anticaries active ingredient comprises calcium fluoride and is present at greater than or equal to about 45% by weight to less than or equal to about 60% by weight after incorporation into each nanoparticle of the plurality of biofilm-targeting nanoparticles, and the matrix-degrading enzyme comprises dextranase.

24. The method of claim 19, wherein the biofilm-targeting nanoparticles are capable of selectively accumulating within a biofilm matrix associated with a surface of the tooth in the oral cavity of the subject.
